(19) United States
(12) Patent Application Publication
(10) Pub. No.: US 2017/0239436 A1
(43) Pub. Date: Aug. 24, 2017

(54) Endotracheal Tube Stylet and Methods of Using the Same

(71) Applicant: Thomas Dinghua Lei, Mountain View, CA (US)

(72) Inventor: Thomas Dinghua Lei, Mountain View, CA (US)

(21) Appl. No.: 15/433,408

(22) Filed: Feb. 15, 2017

Related U.S. Application Data
(60) Provisional application No. 62/298,128, filed on Feb. 22, 2016.

(51) Int. Cl. A61M 16/04

(52) U.S. Cl. CPC A61M 16/0488; A61M 2205/583

(57) ABSTRACT

Endotracheal tube stylets and methods of using the same are provided. Aspects of the stylets include an elongated body having a proximal end and a distal end; an expandable member located at the distal end; and a lumen extending from the expandable member to a fluid port positioned at least near the proximal end. The stylets find use in a variety of different applications.

### CROSS-REFERENCE TO RELATED APPLICATIONS

Pursuant to 35 U.S.C. §119(e), this application claims priority to the filing date of U.S. Provisional Application Ser. No. 62/298,128 filed Feb. 22, 2016, the disclosure of which is herein incorporated by reference.

### INTRODUCTION

Mechanical ventilation is one of the most common life-saving modalities used in modern medicine. Each year, millions of people worldwide receive mechanical ventilation for respiratory support. All major surgeries require mechanical ventilation, either during surgery or for some time shortly after surgery. Millions of critically ill patients cared for at intensive care units (ICU) around world with various medical or surgical conditions, such as acute respiratory failure from different underlying causes or trauma, are also dependent on mechanical ventilation. Of 6,469,674 hospitalizations in the six states surveyed, 180,326 (2.8%) received invasive mechanical ventilation. Projecting to national estimates, there were 790,257 hospitalizations involving mechanical ventilation in 2005, representing 2.7 episodes of mechanical ventilation per 1000 population. Estimated national costs were $27 billion representing 12% of all hospital costs. Mechanical ventilation use is common and accounts for a disproportionate amount of resource use. Quality improvement and cost-reduction strategies are warranted (Wunsch et al., Crit. Care Med. (2010) 38: 1947-1953).

Mechanical ventilation is often achieved either by oral or nasal endotracheal intubation, i.e. placement of an endotracheal tube (ETT) inside the trachea through either the oral cavity or the nasal passage, or in some instances by tracheostomy tube in a chronic or subacute setting, such as with pediatric patients suffering muscular dystrophy or patients of cerebral vascular accidents. Proper placement of an ETT in the trachea is essential to achieve adequate ventilation and mitigate some adverse complications associated with intubation. The current practice is to keep the ETT tip about 2 to 5 cm above from the main carina, i.e., the anatomical structure located at the bifurcation of the trachea. However, malposition of the ETT may occur in up to 10-20% of intubations, with right main bronchus intubation being the most common malposition of the ETT (Mosenifar et al., Practical Pulmonary and Critical Care Medicine (2006) (New York: Taylor & Francis)). Malposition is common due to right main bronchus' anatomical characteristics: shorter, less angular to the trachea. Bronchus intubation results in single lung ventilation of the intubated lung exclusively. The single lung ventilation can cause hypoxic and hypercapneic respiratory failure and can also cause atelectasis of the contralateral lung, in this case, usually the left lung. Unaware of the single lung ventilation, larger tidal volume to the single lung leads to barotrauma and tension pneumothorax at the intubated lung, usually the right lung, or even death (Habgerg et al., Best. Pract. Res. Clin. Anaesthesiol. (2005) 19:641-659). If the ETT is too high from the main carina, it increases risk of accidental self extubation, leads to increased risk of laryngeal and vocal cord injury, and more severely, cardiac arrest, even death if not being recognized in time (Tadié et al., Intensive Care Med. (2010) 36:991-998).

Currently, a post intubation chest X-ray is performed routinely to confirm the ETT position. In addition, while the patient remains on the ventilator, daily chest X-rays are often performed for the sole purpose of checking the ETT position.

Alternatively, as in the operating room, an anesthesiologist often relies solely on clinical observation, which may involve symmetrical chest expansion, oxygenation, air-way pressure and auscultation. However, these imperfect methods regularly lead to inaccurate tube placement, delayed recognition and adverse outcomes. A study of 219 patients showed that while physical examination had an apparent success rate 97%, subsequent chest X-rays of the those “successfully placed ETTs”, showed that 14% required repositioning of the ETT and 5% had a main stem bronchus intubation (Brunel et al., Chest (1989) 96:1043-1045). Bronchoscopy is one alternative to verify the ETT position, however, the added cost of the procedure and the need for a highly trained practitioner are prohibitive and impractical in most surgical patients other than thoracic surgery patients.

There are several technologies that may help to verify proper placement of the ETT. One alternative is a lighted stylet, which uses illumination of the soft tissue of the neck to guide the ETT placement. This technology only provides position with respect to external landmarks, which may not necessarily correlate to the ETT tip location relative to the carina. Additionally, these lighted stylets may not be visible through the soft tissue in some obese patients, and require a light source which adds expense and a power requirement (Hung et al., J. Anaesth. J. Can. Anesth. (1995) 42:820-825). An endotracheal introduction device is known as a gum elastic bougie. As the device is advanced into the patient's trachea the practitioner feels the cartilaginous rings of the trachea and resistance as the bougie engages the carina and/or the small bronchi (Kidd et al., Anaesthesia (1988) 43:437-438. However Shah et al. reports that gum elastic bougies may not be reliable with 20% overall failure rate and 28% failure rate for first time users (Shah et al., J. Emerg. Med. (2011) 41: 429-434).

ICU patients, with prolonged intubation and mechanical ventilation, are at risk of displacement of the ETT. To ensure adequate position, one of the two methods is applied. In the first method, a daily chest x-ray is employed (McGillicuddy et al., Int. J. Emerg. Med. (2009) 2: 247-249). This practice, though it is necessary at times, not only significantly increases total cost of care, but also exposes patients and staff members to substantial levels of radiation. In addition, chest X-ray taking itself in an intubated patient not only entails time, effort, requiring several staff members involvement, adding significant labor cost, but also sometimes leads to undesired adverse events during the process, dislodging supporting devices: such as central intravenous access, nasal or oral gastric tube, even endotracheal tube(ETT) itself accidentally. In the second method, some physicians choose to monitor patients clinically and perform CXR on an as needed basis. This practice by clinical assessment alone, as in the operating room, may not recognize a mal-positioned ETT for days or hours in as high as 15.5% of patients (Schwartz et al., Crit. Care, Med. (1994) 22:1127-1131) leading to contralateral lung hyperinflation (Debnath et al., Indian J. Crit. Care Med. Peer-Rev. Off. Publ. Indian Soc. Crit. Care Med. (2011) 15: 52-54). Bessinger et al. report that 7% of endobronchial intubations were not recognized and not diagnosed until a chest x-ray was conducted (Bissinger et al., Ann. Emerg. Med. (1989) 18: 853-855). So, it is clear that there is a great need to provide a cost-effective and reliable alternative to further improve this common medical procedure: ETT placement and ongoing monitoring of ETT position.

### SUMMARY

Endotracheal tube stylets and methods of using the same are provided. Aspects of the stylets include an elongated body having a proximal end and a distal end; an expandable member located at the distal end; and a lumen extending from the expandable member to a fluid port positioned at least near the proximal end. The stylets find use in a variety of different applications.

In embodiments of the current invention, the device and its mechanisms described here, with malleable property and firmness, maintains the basic function of an ETT stylet to facilitate the endotracheal tube placement, while also allowing for measurement of endotracheal tube tip position relative to the main carina, which is often the reference point. By deploying the expandable member, e.g., in the form of a soft landing complex/balloon, at the distal end of the device, immediately following the intubation, the current invention can provide real time measurement of the ETT tip position relative to the main carina, and secure the endotracheal tube at a desired location in a timely fashion. Embodiments of the current invention provide vital clinical information in a real time fashion, greatly reducing the existing operation cost of taking chest X-rays and/or reducing patient and staff radiation exposure, while eliminating any waiting time. The device may be used at initial intubation, and also can be used on the days following intubation whenever it is necessary or desirable, e.g., when the need to know the ETT tip position arises.

Aspects of the invention include an apparatus and related mechanisms. Embodiments of the invention facilitate intubation, placement of the endotracheal tube, and provide real time ETT tip position relative to the main carina. In using embodiments of the invention, the operator can make adjustments if necessary until the ETT tip is positioned at a desired location, which is about 2-5 cm above the main carina in some instances (Cherng et al., J. Clin. Anesth. (2002) 14:271-274; Reed et al., Acad. Emerg. Med. Off. J. Soc. Acad. Emerg. Med. (1997) 4: 1111-1114).

Stylets according to embodiments of the current invention, and particularly the expandable members thereof, may be configured with soft material to protect the delicacy and fragility of the endobronchial mucosa. The expandable member or landing complex may be made of elastomeric material and inflated with a fluid, e.g., air, at the time of deployment. This air-filled landing complex, a bulb shaped balloon when it is deployed, may be made of similar materials as an endotracheal tube cuff and, just like the ETT cuff, may provide maximal protection to the airway mucosa when it lands and parks at the main carina area.

Embodiments of the current invention are configured to make the endotracheal tube position adjustable relative to the main carina, in a real time fashion to provide this vital clinical information to the operators.

Embodiments of the current invention provide for the substitution of chest X-ray currently required, both at the initial intubation and in the days following intubation while patients remaining on mechanical ventilation. By doing so, embodiments of the current invention significantly reduce the cost of labor and material, cut down radiation exposure to patients and health care personnel, e.g., as compared to existing practice of using chest X-ray.

Embodiments of the current invention provide for meaningful substitution of chest X-ray currently required in practice, and reduce adverse events associated with the taking of chest X-rays in mechanically ventilated patients, such as dislodging life sustaining devices, e.g., a central venous catheter, feeding tube and/or the ETT itself.

Embodiments of the current invention expand the arsenal of tools available to the health care professionals in the field of critical care medicine, surgery and emergency medicine, especially out in the field for EMS personnel. Embodiments of the invention provide a novel, meaningful substitution to the existing practice of using chest X-rays. Embodiments of the invention will have a great impact on the current practice of medicine, and significantly reduce health care costs considering the vast number of patients requiring mechanical ventilation on daily basis worldwide.

As such, dual functional stylets and methods of using the same are provided. Aspects of embodiments of the stylets include a longitude body having an air passageway leading to the distal end of the stylet, where the landing complex/balloon is housed; a house capsule is designed to hold the deflated landing complex in a controlled shape and volume similar to the stylet; an access port at around the deploy point at the proximal end where air could be inflated and deflated, the access port is designed in a way that air could only be injected or pulled out using syringe compressing a spring button to provide safe guard; around the deploy point, are distance marks in the scale of centimeters. The zero mark starts at different spots, is relative to, depending the sizes of endotracheal tube, ET size 6.5/0/32 cm, size 7/0/33 cm, size 7.5/0/34 cm, size 8/0/35 cm, etc. Stylets are malleable, made of material such as aluminum encased in fibroplastic material, such as PVC. The stylets of different sizes (both diameters and lengths) find use in both pediatric and adult patients.

### BRIEF DESCRIPTION OF THE FIGURES

FIG. 1 provides an illustrative view of a device according to an embodiment of the invention, showing the expandable member in an undeployed configuration.

FIG. 2 depicts a cross-sectional view of a device according to an embodiment of the invention with the expandable member in a deployed configuration.

FIG. 3 provides a depiction of a stylet according to an embodiment of the invention, showing the expandable member in the form of a distal end balloon in the deployed (fully inflated) state.

FIG. 4 provides a depiction of an ETT operably coupled to the stylet shown in FIG. 3, where a restraint is associated with the distal end balloon in the deflated state.

FIG. 5 provides a depiction of the operably coupled ETT/stylet of FIG. 4, where a restraint has been removed and the distal end balloon has been deployed.

### DETAILED DESCRIPTION

Endotracheal tube stylets and methods of using the same are provided. Aspects of the stylets include an elongated body having a proximal end and a distal end; an expandable member located at the distal end; and a lumen extending from the expandable member to an access (e.g., fluid) port positioned at least near the proximal end. The stylets find use in a variety of different applications.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Stylets

As summarized above, aspects of the invention include endotracheal tube stylets. As the devices are endotracheal tube stylets, they are stylets that are configured to be used to facilitate intubation with the endotracheal tube, and/or proper positioning with an endotracheal tube, e.g., as described in greater detail below. Aspects of the stylets include the ability to measure the endotracheal tube (ETT) distal tip position relative to the main carina, the reference point, in a real time fashion. In some instances, the stylets are configured to be used to measure the ETT tip position on previously intubated patients on mechanical ventilation.

Stylets of embodiments of the invention are devices that may be viewed as a manifold configured to be operationally placed inside of an endotracheal tube prior to the actual intubation and/or are configured to be used during the intubation process and/or at any time following the initial intubation, e.g., whenever the need to know the position of the endotracheal tube tip arises while patients remain on mechanical ventilation. Stylets as described herein include an elongated body having a proximal and distal end. Stylets of the invention are adjustable, in that they may be manipulated by hand to change shape. In some instances, the stylets include an internal malleable metal component, e.g., aluminum, encased with a fibroplastic material, such as PVC. The stylets may be dimensioned to be placed inside an endotracheal tube, either before intubation or when patients are intubated, when the endotracheal tube has been placed in the trachea of a human. In some instances, the stylets have a length (e.g., as measured by the length of the elongate body thereof) ranging from 25 to 75 cm, such as 30 to 60 cm, including 42 to 46 cm, and a diameter ranging from 10 FR (3.33 mm) to 14 FR (4.67 mm), where in some instances the diameter is 10 FR, 11 FR, 12 FR, 13 FR or 14 FR. The cross-sectional shape of the stylet may vary, where cross-sectional shapes of interest include, but are not limited to: circular, square, trapezoidal, ovoid, etc., where in some instances the cross-sectional shape is circular. Where the stylets are circular in structure, the diameters of the stylets may vary. In some instances, the stylets have an outer diameter ranging from 10 FR to 14 FR, e.g., 10 FR, 11 FR, 12 FR, 13 FR or 14 FR. The dimensions, e.g., length, diameters, along the elongate body may vary or be constant, as desired.

As summarized above, endotracheal tube stylets as described herein include a distal end expandable member, i.e., an expandable member located at the distal end of the stylet. The expandable member may be any structure that can be deployed when desired into a compliant configuration and, in some instances, mate with a human carina. As the expanded member is compliant, it is not rigid but instead is giving or flexible. By mate with a human carina is meant that the expandable structure, when deployed, may touch a human carina in a manner that does not substantially adversely impact the carina, if at all, e.g., in that it causes little if any trauma to the human carina when it touches the human carina. The expandable member may be configured to correspond anatomically to at least a portion of the human carina, as desired. While the dimensions of the expandable member may vary, when present in a fully deployed state, the expandable member may be configured to occupy a volume ranging from 5 to 15 cm³, such as 10 to 12 cm³.

While the expandable member may vary, in some instances the expandable member is a balloon.

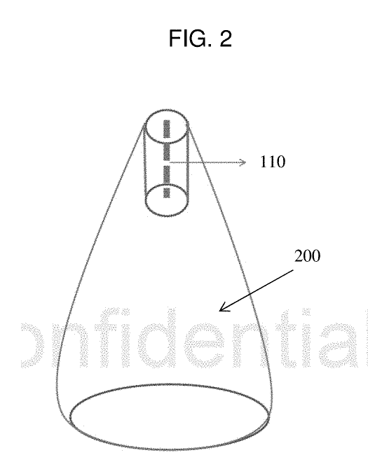

As such, stylets of embodiments of the invention include an expandable member that is an inflatable landing complex/balloon located at the distal tip of the stylet. The landing complex or apparatus (balloon) when inflated, may be configured in such way that it contains an inflatable bulb shaped balloon at the distal end of the stylet, e.g., as shown in FIG. 2. As shown in FIG. 2, inflatable landing complex 200 is fixed to the distal end 110 of the device. While the dimensions of the balloon may vary, in some instances the balloon has a diameter in the deployed (i.e., fully inflated) state ranging from 5 mm (pediatric, 5 mm to 14 mm up to age 16) to 30 mm (adult, 25-27 mm for male, 21-23 mm female). Inflated bulb shaped landing complexes/balloons are, in some instances, configured to safely and securely park at the main carina area, the ground zero or the reference starting point of endotracheal tube tip position. As such, the deployed balloon may be configured to stably associate with the carina in a manner that causes little if any trauma to the carina.

In addition to the distal end expandable member, stylets of the invention may include a lumen that extends from the expandable member to a location at least near the proximal end of the elongated body. By at least near the proximal end of the elongate body is meant that the proximal end of the lumen is located 5 cm or less, such as 3 cm or less from the proximal end of the elongate body, where in some instances the proximal end of the lumen may be located at the proximal end of the elongate body. While the dimensions of the lumen may vary, in some instances the lumen may have an inner diameter ranging from 0.1 to 0.5 mm, such as 0.2 to 0.3 mm. The lumen may be positioned within or on a surface of the elongate body, as desired.

Where the expandable member is a balloon, e.g., as described above, the lumen may serve as a fluidic connector of the balloon to a source of inflation medium, e.g., a liquid or gas, e.g., air, As such, in some instances the stylets are characterized by having a distal end landing complex/balloon that is connected via air passage channel (which is the lumen) extending along the body (and specifically the outer material of the body, such as the casing (which may be made of fibroplastic material, e.g., PVC)) to an access port located at least near the proximal end of the body. The access port may be any fluid port that provides for introduction of a fluid, e.g., gas or liquid, into the lumen and thereby into the balloon located at the distal end of the stylet. The fluid port may be configured in a way that the inflation medium, e.g., air, can only be introduced or removed, i.e., injected or suctioned out, using a syringe. While the fluid port may vary, in some instances the fluid port includes a compressible spring button that provides a safe guard to keep the balloon inflated or deflated, as desired. In these instances, the fluid port of the lumen may be configured to mate with the syringe.

In some instances, the stylets include a scale at a proximal end location. While the proximal end location may vary, in some instances the proximal end location is a deploy point. The proximal end location, e.g., deploy point, is in some instances positioned between 2 to 8, such as 4 to 6 cm form the proximal end of the elongated body. When present, the scale may be on only a portion of the surface of the elongate body, or extend around the elongate body, such that the scale circumscribes the elongate body. As such, in some instances, located around the deploy point of the elongated body are position marks of a scale. The scale and marks thereof may be used to infer the distance of endotracheal tube tip from the main carina when the style is operably coupled to the endotracheal tube. As such, the scale may be configured to provide information about the position of a distal end of an endotracheal tube relative to a carina. The position marks may be triple labeled as summarized above. The scale may be any convenient scale, such as a metric scale (e.g., in centimeters), an English scale (e.g., in inches) etc., as desired. During use, once the stylets are parked securely on the main carina, the endotracheal tube can be slid up and down along the stylet by single operator holding the stylet with one hand, while moving the ETT with another hand before it is finally secured at a desired position.

In some instances, the stylet further includes a removable restraint associated with the expandable member, where the removable restraint is configured to maintain the expandable member in an undeployed configuration. While the removable restraint may vary, in some instances the removable restraint is configured as a cap or analogous structure that fits over the expandable member to maintain the expandable member in an undeployed configuration. The dimensions of the restraint may vary, where in some instances the restraint may have an internal volume ranging from 2 to 8, such as 4 to 6 cm³. In some instances the restraint has color that is different from an endotracheal tube with which the stylet is configured to be used. While the color of the restraint in such embodiments may vary, colors of interest include, but are not limited to: red, green, blue, yellow, brown, orange, black, purple, etc.

The stylets and component parts thereof may be fabricated from any convenient material. In some instances the stylets are malleable, e.g., as described above. In such instances, the stylets may be fabricated from any convenient malleable material(s), where suitable materials include, but are not limited to: medical grade materials such as aluminum encased in a fibroplastic, e.g., PVC, etc. The stylet may be configured as a one time use stylet, where the material from which it is fabricated is chosen in terms of suitability for placement close to, and inside the oral cavity (though no direct contact since it is placed within the ETT, except the landing complex portion) and outside body (in vitro) of a patient and yet be inexpensive enough to provide for one time use.

The stylet may be a sterile, single-use disposable device. The stylets of different sizes find use in either pediatric or adult patients. Choosing a style from a selection of different sized stylets may be not only based on a patients' age, but also on a patient's physical size, in the same manner that a given ETT size is selected. Any other similar system, stylets using different material and techniques for measuring endotracheal tube tip position inside trachea to achieve the similar goal also fall within the scope and spirit of the current invention.

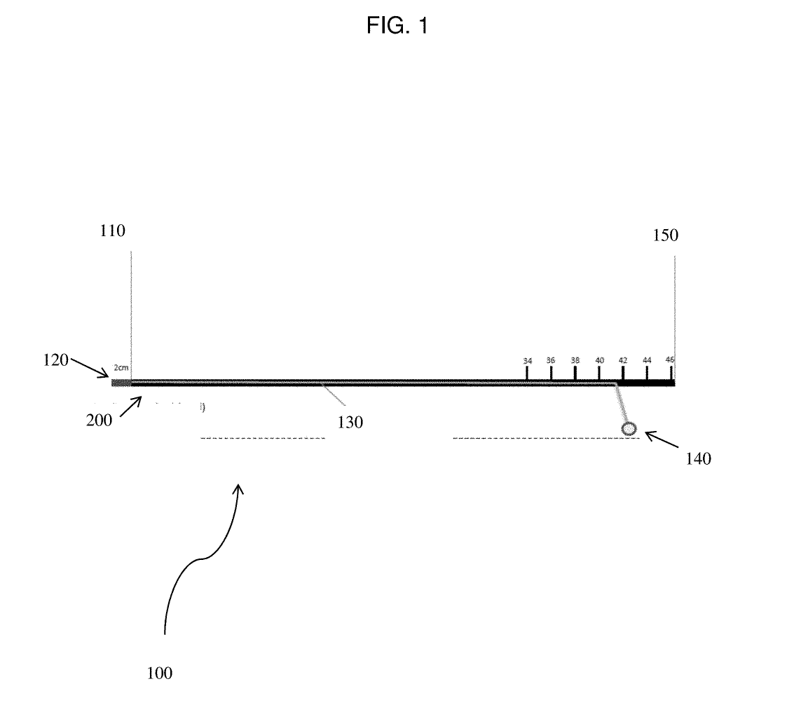

The stylet having been generally described above, a detailed description of a stylet according to an embodiment of the invention is now provided in connection with FIGS. 1 and 2. It is to be understood that the embodiment shown in FIGS. 1 and 2 is merely exemplary of the invention which may be embodied in various forms and sizes using different materials. Therefore, the structure and functional specifics, as well as details presented here are not to be interpreted as limiting and excluding, but merely as the basis for the claims, and as representative basis, while the spirit of the current invention could be employed in various forms and shapes, with appropriate structure details.

As shown in the FIG. 1, the stylet 100 is a cylindrical, tubular body, of various lengths and diameters of choice. Its core is made of malleable metal, such as aluminum, encased with fibroplastic material, such as PVC. At the distal end 110 of the stylet is located an inflatable landing apparatus (balloon) 200 that is encased within a restraint in the form of a cap 120 (which may be colored, e.g., red). The landing complex is connected via an air passage channel (lumen) 130 to the access port (e.g., fluid port) 140 located at around the deploy point near the proximal end 150 the stylet. The access port is configured in a way that air can only be injected or suctioned out using syringe compressing a spring button to provide safe guard and to keep the landing complex either inflated or deflated as desired. The landing apparatus, when inflated, is configured into a bulb shaped balloon, with a diameter ranging from 5 mm (pediatric) to 25 mm (adult). This air inflated bulb shaped landing apparatus can safely and securely park at the main carina area, the ground zero or the starting point of endotracheal tube tip position. Around the deploy point, is a centimeter scale made up of position marks, which are triple labeled as ET size/0/centimeter. During use, the scale provides for the distance of endotracheal tube tip from the main carina to be easily inferred from the distance the ETT moved away/up the reference point.

FIG. 2 shows the distal end expandable member, e.g., landing complex (balloon) 200 of the stylet 100 of FIG. 1 when present in a deployed or inflated state. As shown, the deployed landing complex 200 assumes a bulbous configuration that is configured to mate with the human carina.

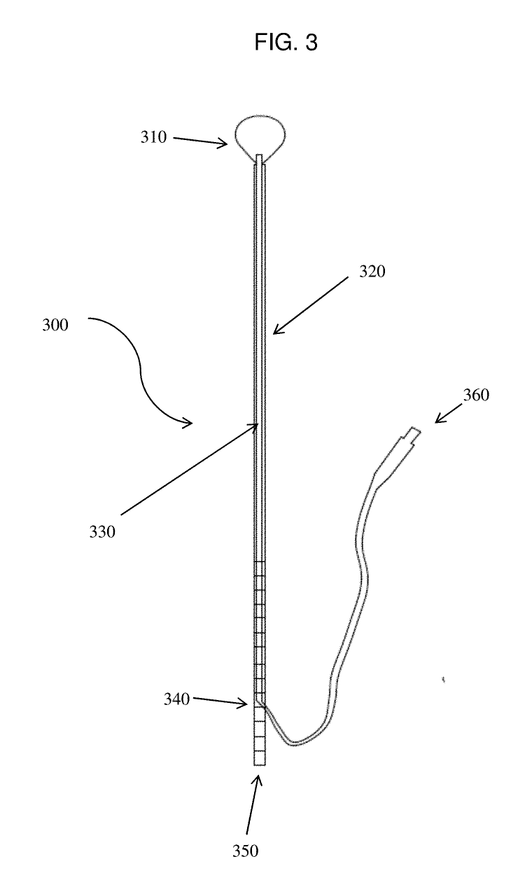

FIG. 3 provides a picture of a stylet 300 according to an embodiment of the invention, showing the expandable member 310 in the form of a distal end balloon in the deployed (fully inflated) state. As shown in FIG. 3, the stylet 300 includes an elongated body 320 with a distal end bulb shaped balloon 310 in the deployed (fully inflated state). Also seen is lumen 330 extending from the distal end balloon 310 along the surface of the stylet to a location 340 near the proximal end 350 of the stylet, e.g., at the deploy point located in the scaled section of the stylet. In the stylet shown in FIG. 3, the lumen separates from the stylet body at the deploy point 340 and continues on to a prefilled syringe 360 to which the access port of the lumen is operably coupled. The prefilled syringe includes a predetermined amount of gas (air) which is sufficient to fully inflate the balloon.

### Methods of Use

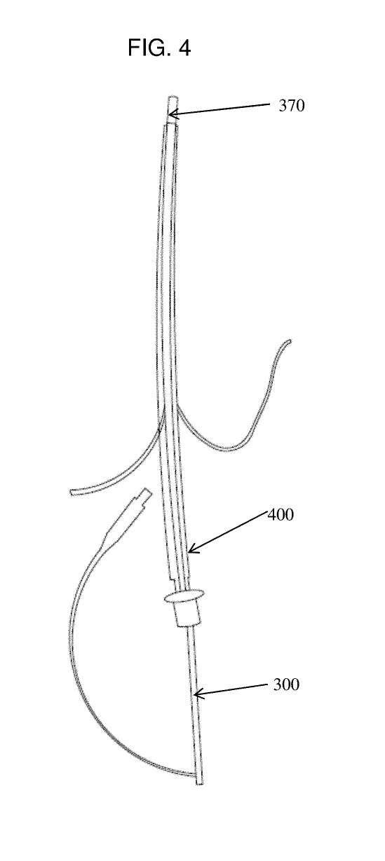

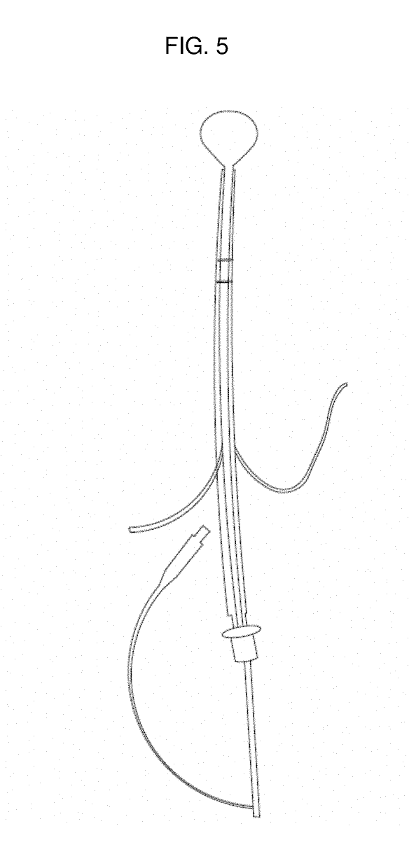

Aspects of the invention further include methods of using the stylets. As indicated above, stylets of the invention may be employed during an initial intubation procedure and/or on patients who are already intubated, where the patients may have been intubated for varying lengths of time, including days or weeks. In methods of invention, when a subject patient is ready to be intubated for initiation of mechanical ventilation, either for elective surgery or due to critical illness requiring ventilator support, as part of preparation for intubation, based on the patient age, physical size, the operator chooses the appropriate sizes of ETT and stylet. The operator then operably couples the selected stylet and ETT. The ETT and stylet may be operably coupled by introducing, e.g., sliding, the selected stylet into the endotracheal tube so that the distal end of the stylet extends all the way to the distal end of the ETT. Where the stylet includes a restraint, such as a red cap, the distal end of the stylet may be extend until the restrain is out of the distal end of the ETT by a suitable distance so that it can be removed, e.g., by a distance ranging from 1 to 2 cm. FIG. 4 provides a picture of an ETT 400 operably coupled to a stylet 300 shown in FIG. 3, where a restraint (cap 370, which may be colored, e.g., red) is associated with the distal end balloon and extends beyond the distal end of the ETT tube. Next, the restraint may be removed and the stylet pulled back so that the expandable member, e.g., the landing complex, is positioned within the distal end of the ETT. The operably associated ETT and stylet may then be employed to intubate a patient as follows. For intubation, the operator places the operably coupled ETT and stylet inside endotracheally and temporarily stops at an estimated, conservative position, e.g. 16 cm or 18 cm, depending on the height of subject patient. As such, the methods include inserting an endotracheal tube operably coupled to stylet into the subject's trachea so that the distal end of the endotracheal tube is separated from the subject's carina by a distance. While the distance between the distal end of the ETT and the carina may vary, in some instances the distance ranges from 4 to 8, such as 5 to 7 cm. Next, the operator slides the stylet back down the ETT about 2 cm. As such, the operator then extends the distal end of the stylet beyond the distal end of the endotracheal tube by a suitable distances to expose the expandable member, where the distance may vary but in some instances ranges from 1 to 5, such as 2 to 3 cm. The operator then deploys the expandable member, e.g., by inflating the landing complex with a preset amount of air through access port via a pre-sized syringe. FIG. 5 provides a picture of the operably coupled ETT/stylet of FIG. 4, where a restraint (370) has been removed and the distal end balloon has been deployed. Next, the operator continues sliding the stylet down the ETT while holding the ETT until the stylet meets certain resistance, whereby the landing complex parks at the main carina area, at this point. In these instances, the distance the stylet has been moved down plus 2 cm indicates the distance of the ETT tip from the main carina. As such, following deployment of the expandable member, the operator advances the stylet until resistance caused by the deployed expandable member meeting the carina is observed. The operator can then make adjustments to the ETT position as desired with one hand holding the stylet, by sliding the ETT up or down along the stylet by another hand to the ideal position (2-5 centimeter). As such, the method may further include approximating the distance of the distal end of the endotracheal tube from the carina by using a scale at the proximal end of the stylet. The ETT may then be secured as desired, e.g., either by tape or ETT holder, at the desired position. Next, the operator deflates the landing apparatus completely, and removes the stylet from ETT. As such, following placement of the ETT, the operator returns the expandable member to an undeployed state and withdraws the stylet from the endotracheal tube.

In patients already on mechanical ventilation, stylets of the invention may be employed to readily check the location of the distal end of the ETT relative to the carina. In such embodiments, the methods include inserting a stylet of the invention into the endotracheal tube so that the distal end of the stylet extends beyond the distal end of the endotracheal tube. If the stylet includes a restraint at the distal end, the restraint is removed before insertion of the stylet into the tube. Following insertion, the expandable member may be deployed, e.g., via inflation, to assume a deployed state. The stylet may then be advanced by a distance until resistance caused by the deployed expandable member meeting the carina is observed. Finally, the distance may be used to determine the location of the distal end of an endotracheal tube relative to the carina in the intubated patient, e.g., using the protocol as described above. Once the location of the distal end of the ETT relative to the carina has been determined, adjustments of the position of the ETT may be made as desired. The stylet may then be removed by returning the expandable member to an undeployed state and withdrawing to the stylet from the endotracheal tube

The subject stylets and methods may be used in a variety of subjects, including humans, e.g., as described above. In certain embodiments, the subjects or patients are humans, ranging from neonates to adults.

### Kits

Also provided are kits for use in practicing the subject methods. The kits at least include a stylet, e.g., as described above. The kits may include one or more additional components that may find use in an application where the stylet is employed, where such additional components include, but are not limited to: a syringe with preset volume (to prevent hyperinflation or under inflation), an endotracheal tube, etc. The stylet (and other components when present) of the kits may be present in a suitable container, such as a sterile container, e.g., a sterile pouch.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, on the packaging of the kit, in a package insert, etc. Yet another form of these instructions is a computer readable medium, e.g., diskette, compact disk (CD), Hard Drive etc., on which the information has been recorded in such form, such as video. Yet another form of these instructions that may be present is a website address which may be used via the internet to access the information at a remote site.

The following examples are put forth so as to provide those of ordinary skill in the art with a complete disclosure and description of how to make the best use of the present invention, and are not intended to limit the scope of what the inventors regard as their invention nor are they intended to represent that the experiments below are all or the only experiments performed.

### EXPERIMENTAL

### I. Initial Intubation Using Dual Functional Stylet

Patient JD is a 68 year old male with a diagnosis of colon cancer. He is scheduled for a planned partial colectomy to completely resect the cancer. The intubation of JD is performed using a style as shown in FIGS. 1 and 2 as follows. The patient is transferred from the pre-surgical department to an operating room. An anesthesiologist starts induction of general anesthesia using either inhaled anesthetics, intravenous anesthetics or both if needed. Once the patient is fully sedated, the anesthesiologist unwraps a single use stylet kit, e.g., as depicted in FIGS. 1 and 2; and an endotracheal tube size of choice. The anesthesiologist slides the style into the ETT all the way to the tip of ETT, removes the red capsule, and pulls back the stylet back into the ETT, to produce an ETT operably coupled to the stylet. The anesthesiologist the places the operably coupled ETT/stylet into the patient's trachea, stops at around 16 cm of ETT mark at patient's lip. The anesthesiologist then slides the stylet down the ETT about 2 cm with one hand while holding the ETT with the other hand, inflates to deploy the landing complex using the preset syringe from the kit, and then continues to slide the stylet down slowly while holding the ETT until it meets resistance and parks at the main carina area. At this point, the distance the stylet moved down plus 2 cm would reflect the distance of the ETT tip from the main carina. The operator then makes adjustments as desired by sliding the ETT up or down along the stylet to the desired position (e.g., 2-5 centimeters). The anesthesiologist deflates the landing apparatus completely and removes the stylet from the ETT. The anesthesiologist then attaches an Easy Cap CO₂ detector to the proximal end of the ETT to confirm endotracheal placement by capnography. Then the anesthesiologist connects the endotracheal tube to a mechanical ventilator directly to start mechanical ventilation. The anesthesiologist secures the endotracheal tube by either taping it or using ETT guard.

### II. Using Stylet to Check ETT Position on Mechanically Ventilated Patient

Ms. JR is a 70 year old female being intubated for respiratory failure. She has been on a ventilator for two days. The respiratory therapist noted the ETT at the lip position moved compared to the flow sheet record from the last shift. The ICU care team wants to know if the ETT is still at adequate position as desired. In order to maintain continuous mechanical ventilation, the respiratory therapist connects the ventilator to the ETT via a three way adaptor, e.g., BETLA. The therapist removes the red capsule from a stylet and slides the stylet through access port of the BETLA Adaptor into the ETT all the way to the full length of the ETT and 2 cm further. The therapist then inflates to deploy the landing complex, continues to slide the stylet down slowly while holding the ETT until the stylet meets resistance and parks at the main carina area. At this point, the distance in centimeters that the style has been slid down plus 2 cm reflects the distance of the ETT tip from the main carina. In this instance, the therapist finds the ETT tip is about 7 cm above the main carina, which is too high and too easy to self extubate (sliding out of patient's trachea). The therapist slides the ETT down 3 cm along the stylet, secures the endotracheal tube by taping it or using ETT guard. The therapist then removes the stylet from the ETT.

The above demonstrates that using a stylet of the invention provides for a noninvasive and real time way to replace the existing practice of either chest X-ray or more complicated/expensive bronchoscopy to confirm ETT position. It gives the operator instant information of ETT position.

Although the foregoing invention has been described in some detail by way of illustration and examples for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. An endotracheal tube stylet, the stylet comprising:
an elongated body having a proximal end and a distal end;
an expandable member located at the distal end; and
a lumen extending from the expandable member to a fluid port positioned at least near the proximal end.

2. The stylet according to claim 1, wherein the expandable member comprises a balloon.

3. The stylet according to claim 2, wherein the balloon is configured to mate with a carina.

4. The stylet according to claim 1, wherein the expandable member occupies a volume ranging from 10 to 12 cm³ in a fully deployed state.

5. The stylet according to claim 1, wherein the stylet further comprises a removable restraint associated with the expandable member, wherein the removable restraint is configured to maintain the expandable member in an undeployed configuration.

6. The stylet according to claim 5, wherein the removable restraint comprises a cap.

7. The stylet according to claim 5, wherein the restraint has color that is different from an endotracheal tube with which the stylet is configured to be used.

8. The stylet according to claim 1, wherein the stylet comprises a scale at a proximal end location.

9. The stylet according to claim 8, wherein the scale is configured to provide information about the position of a distal end of a endotracheal tube relative to a carina.

10. The stylet according to claim 8, wherein the scale is a metric scale.

11. The stylet according to claim 1, wherein the elongated body has a length ranging from 25 to 75 cm.

12. The stylet according to claim 1, wherein the lumen is positioned on a surface of the elongated body.

13. The stylet according to claim 1, wherein the lumen has a diameter ranging from 0.1 to 0.5 mm.

14. The stylet according to claim 1, wherein the fluid port is configured to mate with a syringe.

15. A method of intubating a subject with an endotracheal tube, the method comprising:
(a) inserting an endotracheal tube into the subject's trachea so that the distal end of the endotracheal tube is separated from the subject's carina by a distance, wherein the endotracheal tube is operably coupled to a stylet according to claim 1;
(b) extending the distal end of the stylet beyond the distal end of the endotracheal tube;
(c) deploying the expandable member to assume a deployed state;
(d) advancing the stylet until resistance caused by the deployed expandable member meeting the carina is observed; and
(e) returning the expandable member to an undeployed stated and withdrawing to the stylet from the endotracheal tube;
to intubate the subject with the endotracheal tube.

16. The method according to claim 15, wherein the expandable member comprises a balloon and the method comprises inflating the balloon by introducing a fluid into the fluid port of the lumen.

17. (canceled)

18. The method according to claim 16, wherein the fluid is introduced from a syringe operably coupled to the fluid port.

19. (canceled)

20. The method according to claim 15, wherein the method further comprises operably coupling the endotracheal tube and the stylet.

21. The method according to claim 21, wherein the operably coupling comprises:
(a) introducing the stylet comprising an expandable member removable restraint so that the distal end of the stylet extends beyond the distal end of the endotracheal tube;
(b) removing the removable restraint from the distal end of the stylet; and
(c) withdrawing the stylet so that the distal end of the stylet is within the distal end of the endotracheal tube.

22. (canceled)

23. A method of determining the location of the distal end of an endotracheal tube relative to the carina in an intubated patient, the method comprising:
(a) inserting a stylet into the endotracheal tube so that the distal end of the stylet extends beyond the distal end of the endotracheal tube;
(b) deploying the expandable member to assume a deployed state;
(c) advancing the stylet by a distance until resistance caused by the deployed expandable member meeting the carina is observed; and
(d) using the distance to determine the location of the distal end of an endotracheal tube relative to the carina in the intubated patient.

24-26. (canceled)
